(19) United States
(12) Patent Application Publication
(10) Pub. No.: US 2026/0248510 A1
(43) Pub. Date: Aug. 27, 2026

(54) CIRCULAR FRAME REACTION FORCE SENSING

(71) Applicant: Cilag GmbH International, Cincinnati, OH (US)

(72) Inventors: Matthew HILL, Cincinnati, OH (US); Evan CARRICO, Cincinnati, OH (US); Joseph MURPHY, Cincinnati, OH (US); Mark OVERMYER, Cincinnati, OH (US)

(73) Assignee: Cilag GmbH International, Cincinnati, OH (US)

(21) Appl. No.: 19/545,233

(22) Filed: Feb. 20, 2026

Related U.S. Application Data
(60) Provisional application No. 63/761,667, filed on Feb. 21, 2025.

(51) Int. Cl. A61B 17/115; A61B 17/00; A61B 17/072

(52) U.S. Cl. CPC A61B 17/1155; A61B 17/072; A61B 2017/00075; A61B 2017/07257; A61B 2017/07285

(57) ABSTRACT

A circular stapler for cutting and applying one or more surgical staples to tissue is disclosed. The circular stapler includes various forms of detection systems that are configured to provide feedback to the surgeon that proper tissue compression has been achieved between an anvil of the circular stapler and a stapling head of the circular stapler before firing of the circular stapler. The various forms of detections systems can include one or more of sensors, thin rods, couplers with spur gears, torsion springs, and planetary gears, among other options for detecting that proper tissue compression has been achieved between the anvil and the stapling head before firing of the circular stapler.

### CROSS REFERENCE TO RELATED APPLICATIONS

This application claims the benefit of U.S. Provisional Application No. 63/761,667, filed Feb. 21, 2025, which is incorporated by reference as if fully set forth.

### TECHNICAL FIELD

The present disclosure is directed to circular staplers for use in medical procedures, and more particularly to reaction force sensing on a frame of a circular stapler used in medical procedures.

### BACKGROUND

In certain types of surgical procedures, the use of surgical staples has become the preferred method of joining tissue. Therefore, specially designed surgical staplers have been developed for these applications, which can be referred to as circular staplers in some examples. Circular staplers have become particularly useful for performing an anastomosis procedure, as is known. An anastomosis procedure includes joining sections of intestine of a patient together after a portion of intestine has been removed from the patient. The anastomosis procedure requires re-joining the ends of the two tubular sections together to form a continuous tubular pathway, which is accomplished by the circular stapler. During the surgical procedure, an anvil of the circular stapler must be properly connected to a trocar of the circular stapler to properly perform cutting and stapling of the tissue. Additionally, once the anvil is connected to the trocar, proper compression of the tissue between the anvil and the stapling head is required to perform cutting and stapling of the tissue. If proper tissue compression is not achieved before firing (e.g., cutting and stapling), adverse outcomes and improperly secured tissue may occur. In existing circular staplers, it is difficult for the user (e.g., a surgeon) to determine if proper tissue compression has been achieved.

Therefore, there is a need to provide feedback to the user that proper tissue compression has been achieved before closing and firing of the circular stapler.

### SUMMARY OF THE DISCLOSURE

According to one aspect, a circular stapler can include a housing with a handle extending from the housing, and a shaft coupled to and extending from the housing in a different direction than the handle. A stapling head can be coupled to a distal end of the shaft, and the stapling head can be configured to cut tissue and dispense one or more staples into tissue. An adjustment knob can be rotatably coupled to the housing, and the adjustment knob can be coupled to a first end of a pushrod extending through the housing and the shaft. A trocar can be coupled to a second end of the pushrod, and the trocar can be positioned within and axially translatable relative to the stapling head. An anvil can be removably coupled to a distal end of the trocar. At least one detector can be disposed within or adjacent to the adjustment knob. The at least one detector can be configured to detect an amount of tissue compression of tissue clamped between the anvil and the stapling head prior to firing of the circular stapler.

In one aspect, the at least one detector comprises a force sensor positioned between the adjustment knob and the housing.

In one aspect, a force detected by the force sensor correlates to a tissue compression between the anvil and the stapling head.

In one aspect, the force sensor is a circular resistive or capacitive thin film force sensor.

In one aspect, the force sensor is a plurality of individual force sensors oriented in a circular pattern.

In one aspect, the at least one detector comprises a thin rod that is configured to deflect during rotation of the adjustment knob, and the thin rod deflection correlates to torque experienced by the adjustment knob.

In one aspect, the thin rod is positioned within and axially aligned with the adjustment knob.

In one aspect, the thin rod is positioned radially adjacent to a coupler that is coupled to an axial end of the adjustment knob.

In one aspect, the coupler includes at least one spur gear that is configured to contact the thin rod to deflect the thin rod.

In one aspect, at least one sensor is positioned adjacent to the thin rod, and the at least one sensor is configured to detect the deflection of the thin rod.

In one aspect, the at least one detector comprises a torsion spring positioned within the adjustment knob, and rotation of the adjustment knob causes the torsion spring to bias against an internal surface of the adjustment knob to cause deflection of the torsion spring.

In one aspect, at least one sensor is positioned adjacent to the torsion spring, and the at least one sensor is configured to detect the deflection of the torsion spring.

In one aspect, the at least one detector comprises a planetary gear that is coupled to an adjustment rod, the adjustment rod also being coupled to the adjustment knob at an axial end of the adjustment rod.

In one aspect, rotation of the adjustment knob causes a ring gear of the planetary gear to contact a brake pad coupled to the housing, and friction between the ring gear and the brake pad causes the planetary gear to actuate linearly.

In one aspect, at least one sensor is positioned adjacent to the planetary gear, and the at least one sensor is configured to detect linear movement of the planetary gear.

According to another aspect, a method of operating a circular stapler to staple tissue is provided. The method can include positioning a stapling head of the circular stapler and an anvil removably coupled to a trocar of the circular stapler with tissue therebetween. Rotating an adjustment knob of the circular stapler, the adjustment knob being coupled to a pushrod coupled to the trocar, to axially translate the trocar relative to the stapling head to clamp the tissue between the anvil and the stapling head. Prior to firing the circular stapler, detecting, with at least one detector disposed within or adjacent to the adjustment knob, an amount of tissue compression of the tissue clamped between the anvil and the stapling head. Firing the circular stapler, after detecting the amount of tissue compression, to cut the tissue and dispense one or more staples into the tissue.

In one aspect, the detecting can include measuring, with a force sensor positioned between the adjustment knob and a housing of the circular stapler, a force applied to the adjustment knob, the force correlating to the amount of tissue compression between the anvil and the stapling head.

In one aspect, the detecting can include deflecting a thin rod during rotation of the adjustment knob, the thin rod deflection correlating to torque experienced by the adjustment knob, and detecting the deflection of the thin rod with at least one sensor positioned adjacent to the thin rod.

In one aspect, the detecting can include rotating the adjustment knob to cause a torsion spring positioned within the adjustment knob to bias against an internal surface of the adjustment knob to deflect the torsion spring, and detecting the deflection of the torsion spring with at least one sensor positioned adjacent to the torsion spring.

In one aspect, the detecting can include rotating the adjustment knob to actuate a planetary gear coupled to an adjustment rod that is coupled to the adjustment knob such that a ring gear of the planetary gear contacts a brake pad coupled to a housing of the circular stapler and friction between the ring gear and the brake pad causes linear actuation of the planetary gear, and detecting the linear actuation of the planetary gear with at least one sensor positioned adjacent to the planetary gear.

### BRIEF DESCRIPTION OF THE DRAWINGS

The foregoing Summary as well as the following Detailed Description will be best understood when read in conjunction with the appended drawings, which illustrate exemplary embodiments. In the drawings:

FIG. 1 is a perspective view of an exemplary embodiment of a circular stapler according to the present disclosure.

FIG. 2 is a partial side-view of the circular stapler of FIG. 1.

FIG. 3 is a magnified perspective view of a distal end of the circular stapler of FIG. 1, illustrating an anvil separated from a trocar of the circular stapler.

FIG. 4 is a partial cross-sectional view of a proximal end of the circular stapler of FIG. 1, illustrating an embodiment of a detection system and an adjustment knob of the circular stapler.

FIG. 5 is a partially transparent perspective view of the detection system and the adjustment knob of FIG. 4.

FIG. 6 is a perspective view of an example sensor of the detection system of FIGS. 4-5.

FIG. 7 is a perspective view of another example sensor of the detection system of FIGS. 4-5.

FIG. 8 is a partially transparent perspective view of a proximal end of the circular stapler of FIG. 1, illustrating another embodiment of a detection system and an adjustment knob of the circular stapler.

FIG. 9 is a cross-sectional view of an adjustment knob of another embodiment of a detection system of the circular stapler.

FIG. 10 is a partially transparent perspective view of a proximal end of the circular stapler of FIG. 1, illustrating the detection system and the adjustment knob of FIG. 9.

FIG. 11 is a perspective view of another embodiment of a detection system of the circular stapler.

FIG. 12 is a perspective view of a proximal end of the circular stapler of FIG. 1, illustrating another embodiment of a detection system of the circular stapler.

FIG. 13 is a cross-sectional view of a portion of the detection system of FIG. 12.

FIG. 14 is another cross-sectional view of a portion of the detection system of FIG. 12.

FIG. 15A is a graph illustrating input torque versus knob rotation for the detection system of FIG. 12.

FIG. 15B is a graph illustrating output torque versus knob rotation for the detection system of FIG. 12.

### DETAILED DESCRIPTION

Certain terminology is used in the following description for convenience only and is not limiting. The words “front”, “rear”, “upper”, and “lower” designate directions in the drawings to which reference is made. The words “inwardly” and “outwardly” refer to directions towards and away from parts referenced in the drawings. “Axially” refers to a direction along the axis of a shaft or other cylindrically shaped component. A reference to a list of items that are cited as “at least one of a, b, or c” (where a, b, and c represent the items being listed) means any single one of the items a, b, or c, or combinations thereof. The terms “generally” and “approximately” are to be construed as within 10% of a stated value or ratio, unless otherwise noted. Additionally, the terms “proximal” and “distal” are used with reference to a handle portion of a circular stapler disclosed herein. The term “proximal” referring to the portion closest to the handle portion and the term “distal” referring to the portion located away from the handle portion in a direction of a tip of the circular stapler. The terminology includes the words specifically noted above, derivatives thereof, and words of similar import.

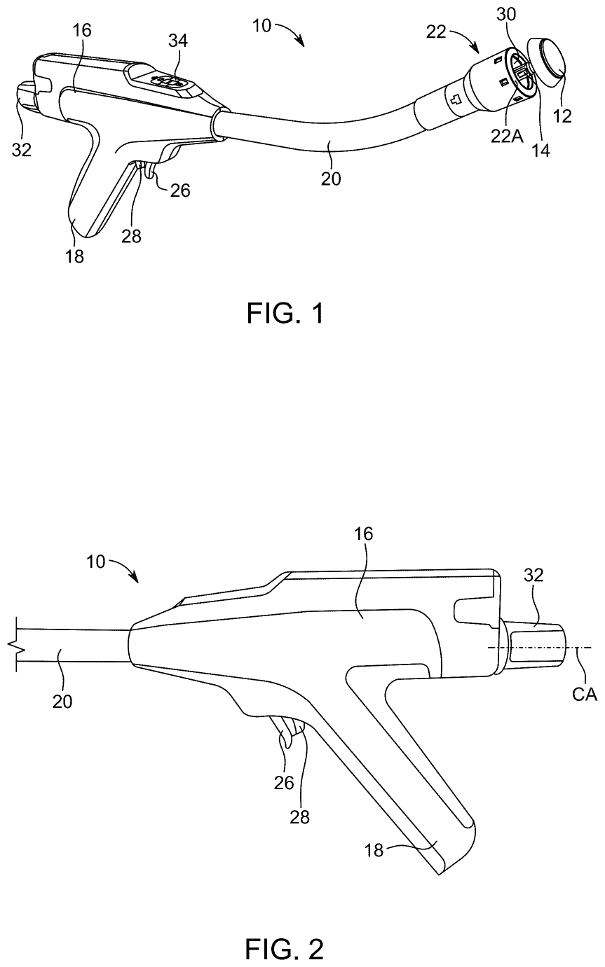

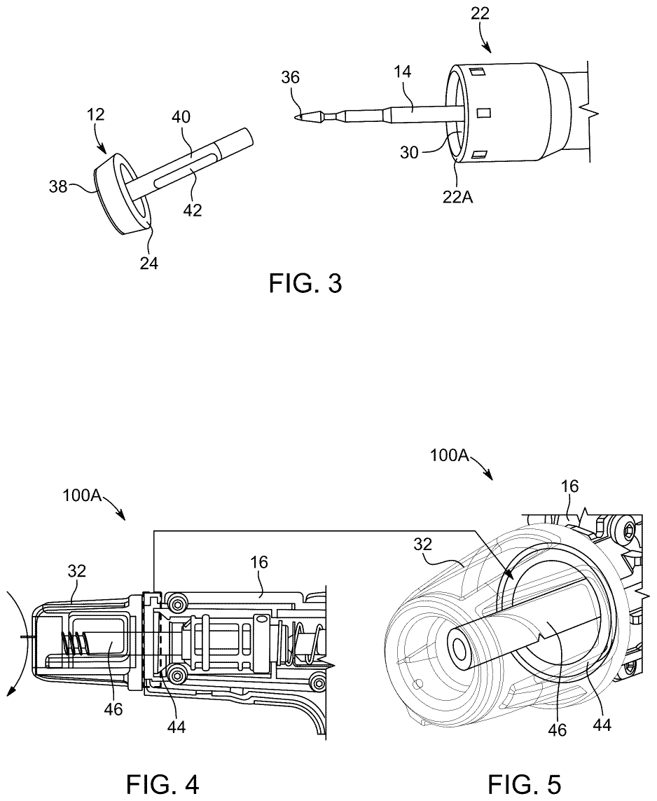

FIG. 1 is a perspective view of an exemplary embodiment of a circular stapler 10 according to the present disclosure. FIG. 2 is a partial side-view of the circular stapler 10. FIG. 3 is a magnified perspective view of a distal end of the circular stapler 10, illustrating an anvil 12 separated from a trocar 14 of the circular stapler 10. FIGS. 1-3 will be discussed together. The circular stapler 10 includes a housing 16 with a handle 18 extending downwards and away from the housing 16, the handle 18 being the feature a user (e.g., a surgeon) grasps while using the circular stapler 10. It is to be understood that the terms “user” and “surgeon” may be used interchangeably throughout the present disclosure, with the intended meaning being the person holding and operating the circular stapler 10. Additionally, it is to be understood that the “user” could be a surgical assistant that is operating the circular stapler 10 based on instructions provided by the surgeon. In other embodiments, the circular stapler 10 can be coupled to or integrated into a robotics system in which a robotic arm supports and operates the circular stapler 10 based on the surgeon's instructions/guidance through a remotely operated graphical user interface (GUI) or other controller communicatively coupled to the robotic arm/system. As such, the circular stapler 10 can be supported and operated by the surgeon, surgical assistant, and/or a robotics arm/system, each of which are controlled or guided by the surgeon.

A shaft 20 extends outwards from the housing 16, with the shaft 20 having the shape of an elongated tube with a circular cross-section. Additionally, the shaft 20 can include a slight bend or curvature, such that the shaft 20 is not a straight tube. A stapling head 22 is positioned at a distal end of the shaft 20, with the distal end of the shaft 20 being the end furthest from the housing 16 of the circular stapler 10. A trocar 14 is positioned within and axially aligned with the stapling head 22, and the trocar 14 is axially translatable relative to the stapling head 22. In other words, the trocar 14 can translate axially inwardly and outwardly relative to the stapling head 22, such that the trocar 14 can extend axially outwards of the stapling head 22 (see FIG. 3) and the trocar 14 can be positioned fully within and surrounded by the stapling head 22. An anvil 12 is removably coupled to the trocar 14, such that the anvil 12 can be connected to and removed from the trocar 14, discussed further below. When the anvil 12 is coupled to the trocar 14, the anvil 12 is axially aligned with the trocar 14 and therefore also axially aligned with the stapling head 22. Additionally, when the anvil 12 is coupled to the trocar 14, the anvil 12 is also axially translatable relative to the stapling head 22. The anvil 12 includes a staple forming surface 24 that is configured to engage with a distal face or surface of the stapling head 22, discussed further below.

A trigger 26 is pivotally coupled to the housing 16 adjacent the handle 18, with the trigger 26 being pivotal relative to the housing 16. Actuation of the trigger 26 causes firing or operation of the circular stapler 10, discussed further below. A safety 28 is coupled to the housing 16 and positioned adjacent the trigger 26, between the trigger 26 and the handle 18. The safety 28 is pivotal relative to the housing 16, and the safety 28 is configured to prevent the trigger 26 from being actuated when in the locked or safe position, and the safety allows the trigger 26 to be actuated when in the released or fire position. The released or fire position being the position in which the safety 28 is pivoted away from the trigger 26, allowing the trigger 26 to be actuated which fires or operates the circular stapler 10. When the trigger 26 is actuated, an internal drive system (not shown) of the circular stapler 10 operates within the shaft 20 to cause staples to be dispensed from the stapling head 22 into forming contact with the staple forming surface 24. Simultaneously, a knife 30 (see FIG. 3), that is operably supported within the stapling head 22, translates axially outwards from the stapling head 22 to cut tissue held within a circumference of the stapling head 22.

The circular stapler 10 also includes an adjustment knob 32 coupled to a proximal end of the housing 16 that is furthest from the stapling head 22 of the circular stapler 10. The adjustment knob 32 is configured to rotate both clockwise and counterclockwise about a central axis CA of the adjustment knob 32. The adjustment knob 32 is connected to the internal drive system (not shown in FIGS. 1-3) within the housing 16 of the circular stapler 10, and the distal end of the internal drive system is connected to the trocar 14 adjacent and within the stapling head 22. Rotation of the adjustment knob 32 causes actuation of the internal drive system within the housing 16 and the shaft 20, which in turn causes the trocar 14 to translate axially inwardly and outwardly relative to the stapling head 22. As such, the user rotates the adjustment knob 32 at a proximal end of the circular stapler 10 to control the axial translation and positioning of the trocar 14 relative to the stapling head 22 at the distal end of the circular stapler 10. The circular stapler 10 can also include a display 34 that is positioned on a top side of the housing 16, opposite the extending direction of the handle 18. The display 34 provides an indication when the tissue compressed between the anvil 12 and the stapling head 22 has reached an appropriate tissue compression, discussed further below.

Referring to FIG. 3, the knife 30 is a circular shaped knife 30 that is positioned within and axially aligned with the stapling head 22. When the trigger 26 is pulled and the internal drive mechanism is actuated (or fired), the knife 30 quickly translates axially outwards from the stapling head 22 and then retracts back into the stapling head 22. The knife 30 is configured to cut tissue that is compressed between the staple forming surface 24 of the anvil 12 and the distal end face 22A of the stapling head 22. The trocar 14 is an elongated metallic shaft with a sharp-pointed distal tip 36 that is configured to cut through tissue. It is to be understood that the trocar 14 can be any surgical instrument with cutting edges at the distal tip 36 for cutting tissue. Additionally, the trocar 14 is shaped and sized to couple to the anvil 12.

The anvil 12 includes an anvil shroud 38 which is a generally circular body portion of the anvil 12. The staple forming surface 24 is formed on the underside of the anvil shroud 38, which underside faces the stapling head 22 when the anvil 12 is coupled to the trocar 14. An anvil shaft 40 extends from the underside of the anvil shroud 38, in a direction axially away from the anvil shroud 38 such that the anvil shaft 40 is axially aligned with the anvil shroud 38. The anvil shaft 40 can have the shape of a hollow or partially hollow elongated cylinder, which shape is a female mating shape to the male mating shape of the trocar 14. Therefore, the anvil shaft 40 and anvil 12 include complimentary shapes and sizes, such that the anvil 12 can be inserted over the distal tip 36 of the trocar 14 to be coupled to and/or removed from the trocar 14. The anvil 12 can further include at least one retaining clip 42 pivotally coupled to the anvil shaft 40. In some examples, the at least one retaining clip 42 can be a leaf-type spring or other spring component that snaps or latches onto features of the trocar 14 to retain the anvil 12 on the trocar 14. Further, in some examples, the at least one retaining clip 42 can be two retaining clips 42 positioned on opposite sides of the anvil shaft 40.

As discussed, the circular stapler 10 includes the elongated shaft 20 and a distal stapling head 22 with a stapling mechanism mounted to the distal end of the shaft 20. The stapling head 22 can also include a stapling cartridge that contains a plurality of staples configured in a concentric circular array. The knife 30 can be concentrically mounted within the stapling cartridge and configured to travel axially within the stapling cartridge. Additionally, the trocar 14 can extend axially from a center of the stapling cartridge, and the trocar 14 is movable relative to the stapling cartridge. The trocar 14 is also adapted to be removably coupled to the anvil 12, as previously discussed. The anvil 12 is configured to form the ends of the staples as they are driven into the staple forming surface 24 of the anvil 12. The distance between a distal face of the staple cartridge and the staple forming surface 24 of the anvil 12 is controlled by adjusting the adjustment knob 32 mounted to the proximal end of the housing 16 of the circular stapler 10. The adjustment knob 32 being configured for controlling the axial movement of the trocar 14. Tissue clamped between the distal end face 22A of the stapling head 22 and the staple forming surface 24 of the anvil 12 is simultaneously stapled and cut when the trigger is actuated by the surgeon.

In some examples, when performing an anastomosis using the circular stapler 10, the intestinal tissue is stapled using double rows of staples being placed on either side of the tissue of the intestine to be removed. The adjoining sections of tissue are simultaneously cut as the adjoining sections of tissue are stapled. In such examples, the surgeon typically inserts the anvil 12 into the proximal end of the lumen (intestinal tissue), proximal of the staple line. This is done by inserting the anvil shroud 38 into an entry port cut into the proximal lumen by the surgeon. In some instances, the anvil 12 can be placed transanally, by placing the anvil shroud 38 on the distal end of the circular stapler 10 and inserting the instrument through the rectum. The surgeon then ties the proximal end of the intestine to the anvil shaft 40 using a suture or other conventional tying device.

Next, the surgeon cuts excess tissue adjacent to the tie and the surgeon attaches the anvil 12 to the trocar 14 of the circular stapler 10. The surgeon then closes the gap between the anvil 12 and stapling head 22 by rotating the adjustment knob 32, thereby retracting the trocar 14 with the attached anvil 12 towards the distal end face 22A of the stapling head. The anvil 12 and trocar 14 are retracted far enough such that the proximal and distal ends of the intestine are clamped in the gap between the staple forming surface 24 and the distal end face 22A of the stapling head 22. During closing of the gap, the surgeon watches the display 34, which indicates when the correct amount of compression has been achieved based on the tissue thickness, among other variables. When the correct compression has been achieved, the surgeon actuates the trigger 26 causing several rows of staples to be driven through both ends of the intestine and formed, thereby joining the ends and forming a tubular pathway. Simultaneously, as the staples are driven and formed, the knife 30 is driven through the intestinal tissue ends, cutting the ends adjacent to the inner row of staples. The surgeon then withdraws the circular stapler 10 from the intestine and the anastomosis procedure is complete.

During the aforementioned stapling and cutting process, it is often difficult for the surgeon to determine if proper tissue compression has been achieved between the distal end face 22A of the stapling head 22 and the staple forming surface 24 of the anvil 12. If proper tissue compression is not achieved before the cutting and stapling, adverse outcomes and improperly secured tissue may occur. In previous circular staplers, it was difficult for the user (e.g., surgeon) to determine if proper tissue compression has been achieved. Circular stapler 10 alleviates the aforementioned issues by providing feedback to the user that proper tissue compression has been achieved before the cutting and stapling by the circular stapler 10. Several different embodiments of detection systems are described in detail below, with each detection system providing an indication to the user that proper tissue compression has been achieved before firing of the circular stapler 10.

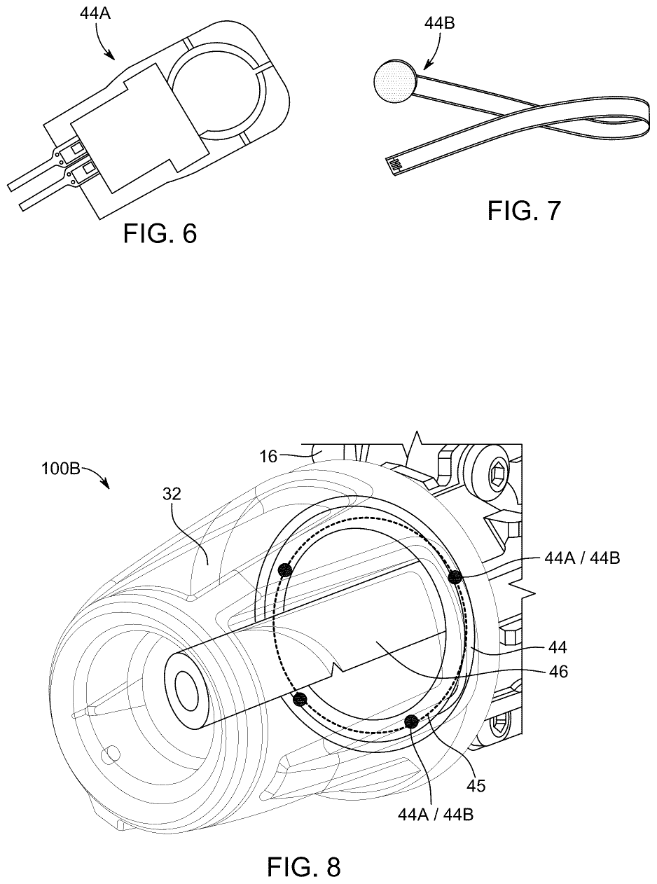

FIG. 4 is a partial cross-sectional view of a proximal end of the circular stapler 10, illustrating an embodiment of a detection system 100A and the adjustment knob 32 of the circular stapler 10. FIG. 5 is a partially transparent perspective view of the detection system 100A and the adjustment knob 32 of FIG. 4. FIG. 6 is a perspective view of an example sensor 44A of the detection system 100A of FIGS. 4-5. FIG. 7 is a perspective view of another example sensor 44B of the detection system 100A of FIGS. 4-5. In some examples, the sensors 44A, 44B can be force sensitive resistors. In other examples, the sensors 44A, 44B may be other sensors other than force sensitive resistors, not specifically listed. FIGS. 4-7 will be discussed together. The detection system 100A is configured to provide an indication to the user that proper tissue compression has been achieved between the distal end face 22A of the stapling head 22 and the staple forming surface 24 of the anvil 12, before firing of the circular stapler 10.

In the detection system 100A, a sensor 44 is positioned between an end of the adjustment knob 32 facing the housing 16 and an end of the housing 16 facing the adjustment knob 32. The sensor 44 can be a force sensor that is configured to capture the reaction force associated with closing or retracting the trocar 14 of the circular stapler 10, which is achieved by rotating the adjustment knob 32 clockwise such that the adjustment knob 32 axially moves towards the housing 16. More specifically, clockwise rotation of the adjustment knob 32 relative to the housing 16, causes the adjustment knob 32 to thread towards and translate axially towards the housing 16, which causes the trocar 14 to be retracted into the stapling head 22. Therefore, counterclockwise rotation of the adjustment knob 32 relative to the housing 16, causes the adjustment knob 32 to thread away from and translate axially away from the housing 16, which causes the trocar 14 to be extended outwards from the stapling head 22. As such, the adjustment knob 32 can be described as a lead-nut in a closure mechanism of the circular stapler 10.

When the adjustment knob 32 is rotated clockwise the adjustment knob 32 contacts the housing 16, and further clockwise rotation of the adjustment knob 32 causes the adjustment knob 32 to compress against the housing 16. More specifically, when the adjustment knob 32 is rotated clockwise, external threads on the pushrod 46 engage with internal threads of the adjustment knob 32, which causes the adjustment knob 32 to translate inwards and compress against the housing 16. The sensor 44 is positioned between the adjustment knob 32 and the housing 16, and the sensor 44 is configured to sense and/or record the reaction force between the adjustment knob 32 and the housing 16. As such, the sensor 44 is configured to capture the reaction force associated with closing of the circular stapler 10. The sensed force between the adjustment knob 32 and the housing 16 can then be transferred to a printed circuit board (PCB) or other controller positioned within the housing 16 or positioned remotely from the circular stapler 10 (e.g., a computer or other controller communicatively coupled to the circular stapler 10). The transferred reaction force data can then be analyzed by the controller to approximate the compression force experienced by the tissue clamped between the distal end face 22A of the stapling head 22 and the staple forming surface 24 of the anvil 12 (see FIG. 3). The compression force approximation can be achieved based on previous test results that correlate force produced by the adjustment knob 32 to compression force experienced by the tissue. The compression force can then be presented to the user on the display 34 of the circular stapler 10 and/or on a display of a remotely connected computer/controller.

FIG. 6 is a perspective view of an example sensor 44A of the detection system 100A of FIGS. 4-5. FIG. 7 is a perspective view of another example sensor 44B of the detection system 100A of FIGS. 4-5. The sensor 44A of FIG. 6 is a thin film resistive force sensor that can be used to detect the reaction force between the adjustment knob 32 and the housing 16. The sensor 44B of FIG. 7 is a capacitive force sensor that also can be used to detect the reaction force between the adjustment knob 32 and the housing 16. Additionally, it is to be understood that other non-disclosed force sensors can be utilized as long as the sensor can detect the reaction force between the adjustment knob 32 and the housing 16. In some examples, as illustrated in FIG. 5, the sensor 44 can be a donut or ring shaped sensor having a generally flat-circular cross section. The sensor 44 can be a resistive or capacitive thin film load/force sensor that is shaped and sized to conform to a diameter of the adjustment knob 32.

FIG. 8 is a partially transparent perspective view of a proximal end of the circular stapler 10, illustrating another embodiment of a detection system 100B and the adjustment knob 32 of the circular stapler 10. The detection system 100B is substantially similar to the detection system 100A illustrated in FIGS. 4-5, and it is to be understood that the disclosure regarding detection system 100A equally applies to the detection system 100B unless otherwise noted. Therefore, to avoid redundancy, only the differences between the detection system 100A and the detection system 100B are disclosed below. Additionally, it is to be understood that the sensors 44A, 44B of FIGS. 6 and 7, respectively, can be utilized in the detection system 100B.

The detection system 100B includes a plurality of small sensors 44A, 44B that are positioned about a circular contact area 45 between the adjustment knob 32 and the housing 16. In other words, the sensors 44A, 44B are positioned on the same outer diameter of the circular contact area 45 such that they generally form the shape of a circle. The plurality of small sensors 44A, 44B are configured to mitigate the impact of a user inadvertently pushing on the adjustment knob 32 in a transverse or lateral direction, relative to a central axis of the adjustment knob 32, while rotating the adjustment knob 32. The inadvertent pushing of the adjustment knob 32 in a transverse or lateral direction could alter the force measurements gathered by the sensors 44A, 44B. Therefore, the plurality of small sensors 44A, 44B are configured to gather a plurality of force measurements about the circular contact area 45 between the adjustment knob 32 and the housing 16 to ensure accurate and consistent measurements are being gathered about the circular contact area 45. The gathered reaction force data can then be analyzed by the controller to approximate the compression force experienced by the tissue clamped between the distal end face 22A of the stapling head 22 and the staple forming surface 24 of the anvil 12. The compression force can then be presented to the user on the display 34 of the circular stapler 10 and/or on a display of a remotely connected computer/controller. It is to be understood that in some examples the detection system 100B can include a plurality of small individual sensors 44A, 44B arranged about a circular contact area 45, while in other examples the sensor could be the donut or ring shaped sensor 44 as shown in both FIGS. 5 and 8.

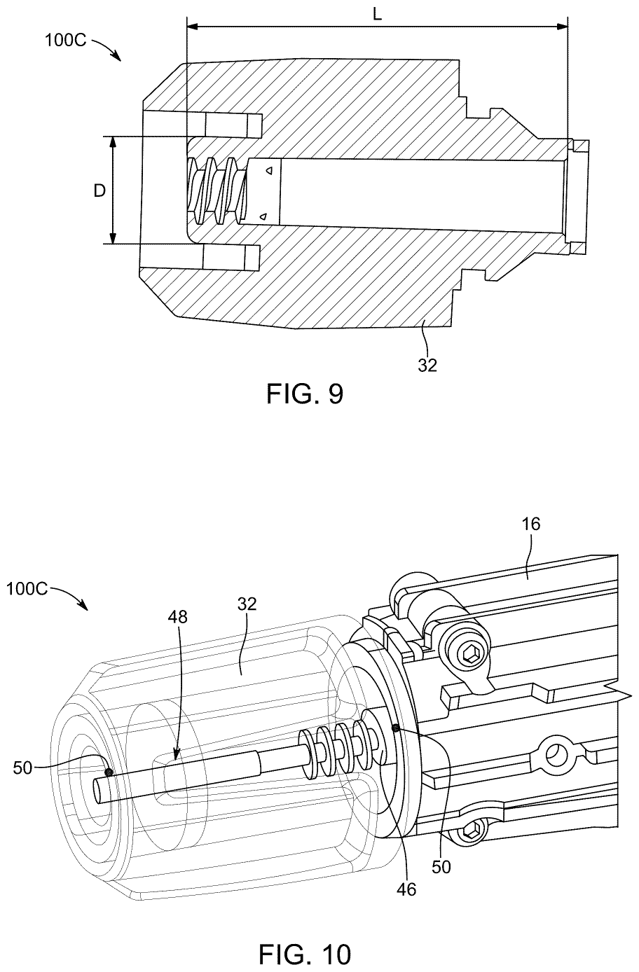

FIG. 9 is a cross-sectional view of the adjustment knob 32 of another embodiment of a detection system 100C of the circular stapler 10. FIG. 10 is a partially transparent perspective view of a proximal end of the circular stapler 10, illustrating the detection system 100C and the adjustment knob 32 of FIG. 9. FIGS. 9-10 will be discussed together. The detection system 100C is configured to provide an indication to the user that proper tissue compression has been achieved between the distal end face 22A of the stapling head 22 and the staple forming surface 24 of the anvil 12, before firing of the circular stapler 10. Specifically, the detection system 100C is configured to detect the torsional strain across the axial length of the adjustment knob 32, which can be amplified and detected by a thin rod 48 coupled and positioned within the adjustment knob 32.

The angular change of the thin rod 48 can be sensed and then used to estimate the amount of torsion placed on the adjustment knob 32. The torsional strain on the adjustment knob 32 can then be analyzed by the controller to approximate the compression force experienced by the tissue clamped between the distal end face 22A of the stapling head 22 and the staple forming surface 24 of the anvil 12. The approximation of the compression can be achieved based on previous test results that correlate torsional strain of the adjustment knob 32 to compression force experienced by the tissue. As illustrated, in FIG. 10 the thin rod 48 can be integrated into the adjustment knob 32 in series with a torsional load, such that the thin rod 48 is axially aligned with the adjustment knob 32 and the pushrod 46. An encoder 50 can be coupled within the proximal and distal ends of the adjustment knob 32, and the encoders 50 can be configured to detect the angular change of the thin rod 48 during rotation of the adjustment knob 32.

The torsional strain experienced by the adjustment knob 32 can be quantified by the equation

$$θ = \frac{TL}{GJ} ,$$

which can be rearranged to be

$$T = \frac{θ GJ}{L} .$$

In the aforementioned equations, “T” represents the torque experienced by the adjustment knob 32. “6” represents the angular deflection of the thin rod 48, which can be multiplied by a known constant or value to represent the angular deflection of the adjustment knob 32. “L” represents the length of the adjustment knob 32 (see FIG. 9), and “G” represents the modulus of rigidity of the adjustment knob 32, which is a known value. Additionally, “J” represents the polar moment of inertia, which can be calculated by the equation

$$J = \frac{{π D}^{4}}{32}$$

for a circular cross-section, with “D” being the internal diameter of the adjustment knob 32, as shown in FIG. 9.

Therefore, the angular deflection of the thin rod 48 can be detected by the encoders 50, which value can be transferred to the PCB or other controller to be multiplied by a known constant or value to represent the angular deflection of the adjustment knob 32. Additionally, the inner diameter “D” of the adjustment knob 32 and the length “L” of the adjustment knob 32 are known values which can be used to calculate the polar moment of inertia and then the torque experienced by the adjustment knob 32, using the aforementioned equations. With the torque experienced by the adjustment knob 32 being calculated, the data can then be analyzed by the controller to approximate the compression force experienced by the tissue clamped between the distal end face 22A of the stapling head 22 and the staple forming surface 24 of the anvil 12. The compression force can then be presented to the user on the display 34 of the circular stapler 10 and/or on a display of a remotely connected computer/controller.

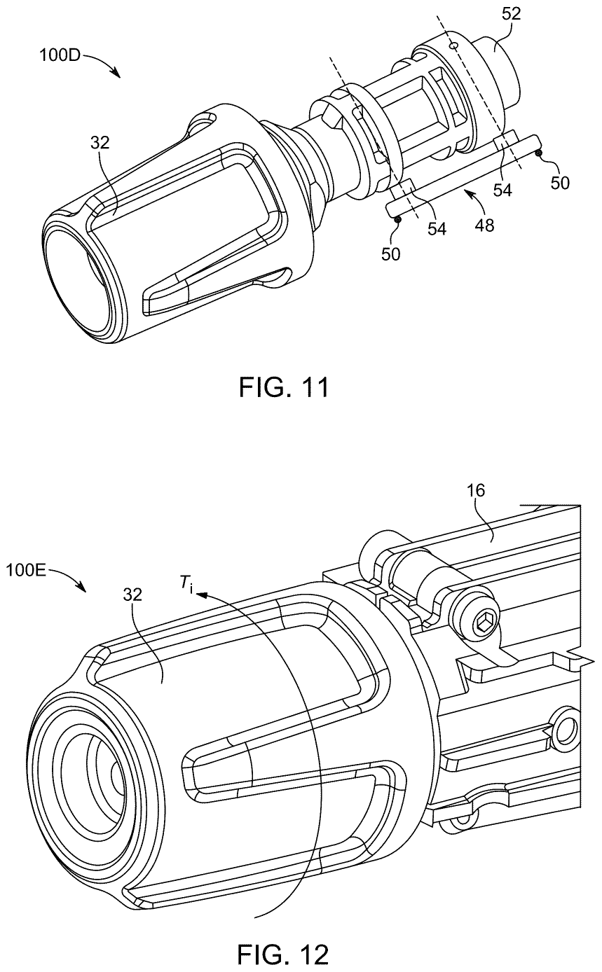

FIG. 11 is a perspective view of another embodiment of a detection system 100D and the adjustment knob 32 of the circular stapler 10. The detection system 100D is substantially similar to the detection system 100C illustrated in FIG. 10, and it is to be understood that the disclosure regarding the detection system 100C equally applies to the detection system 100D unless otherwise noted. Therefore, to avoid redundancy, only the differences between the detection system 100C and the detection system 100D are disclosed below. Additionally, it is to be understood that the housing 16 of the circular stapler 10 is not illustrated in FIG. 11 for clarity purposes, allowing for a clear illustration of the components of the detection system 100D. With that said, it is also to be understood that the detection system 100D can be positioned at least partially within the housing 16, similar to the detection system 100C illustrated in FIG. 10.

As illustrated in FIG. 11, the adjustment knob 32 can be axially aligned with and coupled to a coupler 52 which includes spur gears 54 positioned adjacent each axially end of the coupler 52 on an outer circumference of the coupler 52. Further, the thin rod 48 in the detection system 100D can be positioned parallel with but radially offset from the coupler 52, such that the thin rod 48 is not axially aligned with the coupler 52. Rather, the thin rod 48 is positioned adjacent the outer circumference of the coupler 52, such that the thin rod 48 is configured to contact the spur gears 54 positioned at each axial end of the coupler 52. The thin rod 48 is also configured to angularly deflect based on the rotation of the adjustment knob 32 and the coupler 52, which spur gears 54 of the coupler 52 contact and angularly deform or deflect the thin rod 48.

Specifically, the angular deflection of the thin rod 48 can be detected by the encoders 50, which value can be transferred to the PCB or other controller to be multiplied by a known constant or value to represent the angular deflection and torque experienced by the adjustment knob 32, as previously discussed. With the torque experienced by the adjustment knob 32 being calculated, the data can then be analyzed by the controller to approximate the compression force experienced by the tissue clamped between the distal end face 22A of the stapling head 22 and the staple forming surface 24 of the anvil 12. The compression force can then be presented to the user on the display 34 of the circular stapler 10 and/or on a display of a remotely connected computer/controller. The detection system 100D provides another system that indicates to the user that proper tissue compression has been achieved between the distal end face 22A of the stapling head 22 and the staple forming surface 24 of the anvil 12, before firing of the circular stapler 10.

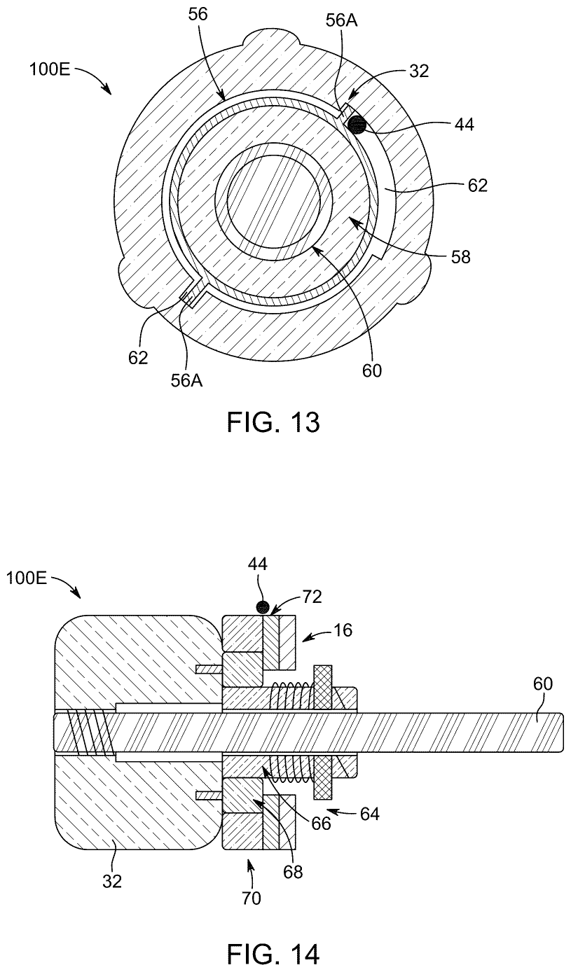

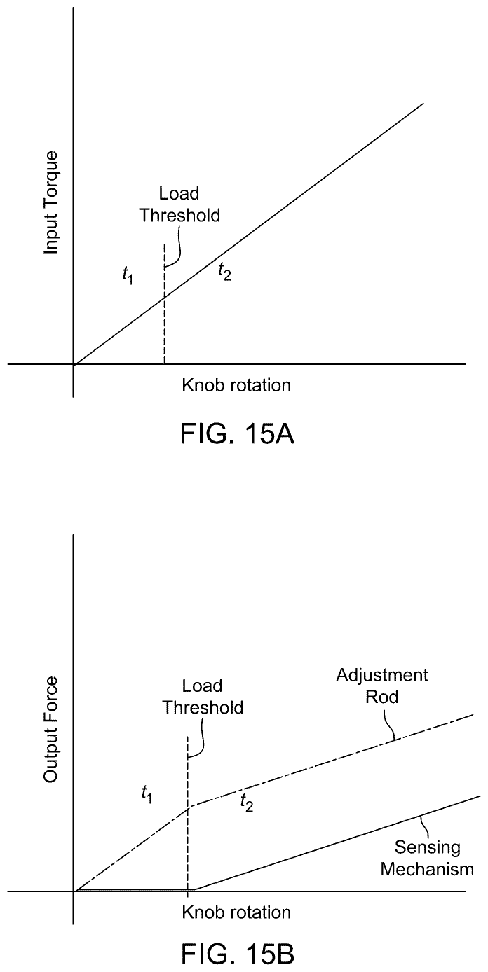

FIG. 12 is a perspective view of a proximal end of the circular stapler 10, illustrating another embodiment of a detection system 100E of the circular stapler 10. FIG. 13 is a cross-sectional view of a portion of an embodiment of the detection system 100E of FIG. 12. FIG. 14 is another cross-sectional view of a portion of another embodiment of the detection system 100E of FIG. 12. FIG. 15A is a graph illustrating input torque versus knob rotation for the detection system 100E. FIG. 15B is a graph illustrating output torque versus knob rotation for the detection system 100E. FIGS. 12-15B will be discussed together. The detection system 100E provides another system that indicates to the user that proper tissue compression has been achieved between the distal end face 22A of the stapling head 22 and the staple forming surface 24 of the anvil 12, before firing of the circular stapler 10.

In general, the detection system 100E is based on a concept in which the torsional force that is applied to the adjustment knob 32 is shared between the adjustment knob 32 and a mechanism including a compliant member positioned within or coupled to the adjustment knob 32, as discussed below with reference to FIGS. 13-14. Additionally, the mechanism including the compliant member is positioned in series with the adjustment knob 32, such that after a predefined load threshold is achieved the torsional load is shared between an adjustment rod connected to the adjustment knob 32 and the mechanism including the compliant member. A sensor 44 (i.e., an encoder) positioned on the compliant member is used to detect deformation and then correlate with the torque applied to the adjustment knob 32. A first embodiment and a second embodiment of the detection system 100E are disclosed below with reference to FIGS. 13-14, respectively.

Referring to FIG. 13, the first embodiment of the detection system 100E includes a torsion spring 56, a driving nut 58, and an adjustment rod 60 positioned within the adjustment knob 32. Specifically, the adjustment rod 60 can be the radially innermost component with the driving nut 58 positioned radially outwards of the adjustment rod 60, and the torsion spring 56 is positioned radially outwards of the driving nut 58. The torsion spring 56 can include at least one radially extending portion 56A that is positioned at least partially within a groove 62 within an inner surface of the adjustment knob 32. The torsion spring 56 is configured to be biased against the adjustment knob 32, such that after a predetermined torsional load is achieved the torsion spring 56 will deflect which is measured by the sensor 44 (i.e., an encoder) positioned within the adjustment knob 32.

The mechanism including the compliant member in the first embodiment illustrated in FIG. 13 is configured to convert input torque to radial motion after the predetermined torsional load is achieved on the adjustment knob 32. The sensor 44 is then used to detect deformation and then correlate with the torque applied to the adjustment knob 32. With the torque experienced by the adjustment knob 32 being calculated, the data can then be analyzed by the controller to approximate the compression force experienced by the tissue clamped between the distal end face 22A of the stapling head 22 and the staple forming surface 24 of the anvil 12. The compression force can then be presented to the user on the display 34 of the circular stapler 10 and/or on a display of a remotely connected computer/controller. The detection system 100E provides another system that indicates to the user that proper tissue compression has been achieved between the distal end face 22A of the stapling head 22 and the staple forming surface 24 of the anvil 12, before firing of the circular stapler 10.

Referring to FIG. 14, the second embodiment of the detection system 100E includes an adjustment rod 60 axially aligned with and coupled at a first end to the adjustment knob 32. A planetary gear 64 includes a sun gear 66 that is axially aligned with and positioned radially outwards of and coupled to the adjustment rod 60. At least one planet gear 68 is positioned radially outwards of the sun gear 66, and the planet gear 68 is operatively coupled to the sun gear 66 such that the at least one planet gear 68 is rotatable relative to the sun gear 66. A ring gear 70 is positioned radially outwards of the at least one planet gear 68, and the ring gear 70 is operatively coupled to the planet gear 68. Additionally, the ring gear 70 can contact a brake pad 72 that is positioned axially between an axial end face of the ring gear 70 and an oppositely facing end face of a portion of the housing 16.

In use, the planetary gear 64 including the sun gear 66, at least one planet gear 68, and ring gear 70 is oriented in series with the adjustment knob 32, such that the planetary gear 64 is overall orientated axially aligned with the adjustment knob 32. Additionally, the ring gear 70 of the planetary gear 64 is configured to interface with the brake pad 72, which can be described in some embodiments as a friction brake that is coupled to and oriented on the housing 16. With the adjustment knob 32 being rotated, a predetermined frictional force between the ring gear 70 and the brake pad 72 will eventually be exceeded, which causes the planetary gear 64 to turn and a separate rod or feature of the sun gear 66 actuates linearly via an internal or external threaded surface on the sun gear 66. As such, the mechanism including the compliant member in the second embodiment illustrated in FIG. 14 is configured to convert input torque to linear motion after the predetermined torsional load is achieved on the adjustment knob 32.

A sensor 44 (i.e., a linear sensor) can then used to detect deformation and then correlate with the torque applied to the adjustment knob 32. With the torque experienced by the adjustment knob 32 being calculated, the data can then be analyzed by the controller to approximate the compression force experienced by the tissue clamped between the distal end face 22A of the stapling head 22 and the staple forming surface 24 of the anvil 12. The compression force can then be presented to the user on the display 34 of the circular stapler 10 and/or on a display of a remotely connected computer/controller. The detection system 100E provides another system that indicates to the user that proper tissue compression has been achieved between the distal end face 22A of the stapling head 22 and the staple forming surface 24 of the anvil 12, before firing of the circular stapler 10.

FIG. 15A is a graph illustrating input torque versus knob rotation for the detection system 100E of FIG. 12. FIG. 15B is a graph illustrating output torque versus knob rotation for the detection system 100E of FIG. 12. FIGS. 12-15B will be discussed together. As illustrated in both FIGS. 15A-15B, “t₁” represents when all torsion applied to the adjustment knob 32 is resolved through the adjustment rod 60 before a load threshold is achieved. Further, as illustrated in both FIGS. 15A-15B, “t₂” represents after the load threshold has been achieved, in which the input torque is shared between the adjustment rod 60 and the compliant mechanism with integrated sensors (i.e., torsion spring 56 and/or planetary gear 64 and brake pad 72). As illustrated in FIG. 15B, once the load threshold is achieved, the torque is shared between the adjustment rod 60 and the sensing mechanism, which deflection or deformation is then sensed by the sensor 44 (i.e., encoders) which is then used to correlate with the torque applied to the adjustment knob 32, as previously discussed. The detection system 100E provides another system that indicates to the user that proper tissue compression has been achieved between the distal end face 22A of the stapling head 22 and the staple forming surface 24 of the anvil 12, before firing of the circular stapler 10.

The circular stapler 10 of the present disclosure alleviates the issues of determining whether the proper tissue compression has been achieved by providing feedback to the surgeon that proper tissue compression has been achieved before firing of the circular stapler 10. Several different embodiments of detection systems are provided, with each detection system providing an indication to the surgeon that proper tissue compression has been achieved before firing of the circular stapler 10. As will be appreciated by those having skill in the art, the circular stapler 10 of the present disclosure provides many advantages over previously known circular staplers.

Having thus described the present embodiments in detail, it is to be appreciated and will be apparent to those skilled in the art that many physical changes, only a few of which are exemplified in the detailed description of the disclosure, could be made without altering the inventive concepts and principles embodied therein. It is also to be appreciated that numerous embodiments incorporating only part of the preferred embodiment are possible which do not alter, with respect to those parts, the inventive concepts and principles embodied therein.

The present embodiment and optional configurations are therefore to be considered in all respects as exemplary and/or illustrative and not restrictive, the scope of the disclosure being indicated by the appended claims rather than by the foregoing description, and all alternate embodiments and changes to this embodiment which come within the meaning and range of equivalency of said claims are therefore to be embraced therein.

What is claimed is:

1. A circular stapler comprising:
a housing with a handle extending from the housing;
a shaft coupled to and extending from the housing in a different direction than the handle;
a stapling head coupled to a distal end of the shaft, the stapling head being configured to cut tissue and dispense one or more staples into tissue;
an adjustment knob rotatably coupled to the housing, the adjustment knob being coupled to a first end of a pushrod extending through the housing and the shaft;
a trocar coupled to a second end of the pushrod, the trocar being positioned within and axially translatable relative to the stapling head;
an anvil removably coupled to a distal end of the trocar; and
at least one detector disposed within or adjacent to the adjustment knob, wherein the at least one detector is configured to detect an amount of tissue compression of tissue clamped between the anvil and the stapling head prior to firing of the circular stapler.

2. The circular stapler according to claim 1, wherein the at least one detector comprises a force sensor positioned between the adjustment knob and the housing.

3. The circular stapler according to claim 2, wherein a force detected by the force sensor correlates to a tissue compression between the anvil and the stapling head.

4. The circular stapler according to claim 2, wherein the force sensor is a circular resistive or capacitive thin film force sensor.

5. The circular stapler according to claim 2, wherein the force sensor is a plurality of individual force sensors oriented in a circular pattern.

6. The circular stapler according to claim 1, wherein the at least one detector comprises a thin rod that is configured to deflect during rotation of the adjustment knob, wherein the thin rod deflection correlates to torque experienced by the adjustment knob.

7. The circular stapler according to claim 6, wherein the thin rod is positioned within and axially aligned with the adjustment knob.

8. The circular stapler according to claim 6, wherein the thin rod is positioned radially adjacent to a coupler that is coupled to an axial end of the adjustment knob.

9. The circular stapler according to claim 8, wherein the coupler includes at least one spur gear that is configured to contact the thin rod to deflect the thin rod.

10. The circular stapler according to claim 6, wherein the at least one detector further comprises at least one sensor positioned adjacent to the thin rod, and wherein the at least one sensor is configured to detect the deflection of the thin rod.

11. The circular stapler according to claim 1, wherein the at least one detector comprises a torsion spring positioned within the adjustment knob, wherein rotation of the adjustment knob causes the torsion spring to bias against an internal surface of the adjustment knob to cause deflection of the torsion spring.

12. The circular stapler according to claim 11, wherein at least one sensor is positioned adjacent to the torsion spring, and wherein the at least one sensor is configured to detect the deflection of the torsion spring.

13. The circular stapler according to claim 1, wherein the at least one detector comprises a planetary gear that is coupled to an adjustment rod, the adjustment rod also being coupled to the adjustment knob at an axial end of the adjustment rod.

14. The circular stapler according to claim 13, wherein rotation of the adjustment knob causes a ring gear of the planetary gear to contact a brake pad coupled to the housing, and wherein friction between the ring gear and the brake pad causes the planetary gear to actuate linearly.

15. The circular stapler according to claim 14, wherein at least one sensor is positioned adjacent to the planetary gear, and wherein the at least one sensor is configured to detect linear movement of the planetary gear.

16. A method of operating a circular stapler to staple tissue, the method comprising:
positioning a stapling head of the circular stapler and an anvil removably coupled to a trocar of the circular stapler with tissue therebetween;
rotating an adjustment knob of the circular stapler, the adjustment knob being coupled to a pushrod coupled to the trocar, to axially translate the trocar relative to the stapling head to clamp the tissue between the anvil and the stapling head;
prior to firing the circular stapler, detecting, with at least one detector disposed within or adjacent to the adjustment knob, an amount of tissue compression of the tissue clamped between the anvil and the stapling head; and
firing the circular stapler, after detecting the amount of tissue compression, to cut the tissue and dispense one or more staples into the tissue.

17. The method according to claim 16, wherein detecting comprises measuring, with a force sensor positioned between the adjustment knob and a housing of the circular stapler, a force applied to the adjustment knob, the force correlating to the amount of tissue compression between the anvil and the stapling head.

18. The method according to claim 16, wherein detecting comprises deflecting a thin rod during rotation of the adjustment knob, the thin rod deflection correlating to torque experienced by the adjustment knob, and detecting the deflection of the thin rod with at least one sensor positioned adjacent to the thin rod.

19. The method according to claim 16, wherein detecting comprises rotating the adjustment knob to cause a torsion spring positioned within the adjustment knob to bias against an internal surface of the adjustment knob to deflect the torsion spring, and detecting the deflection of the torsion spring with at least one sensor positioned adjacent to the torsion spring.

20. The method according to claim 16, wherein detecting comprises rotating the adjustment knob to actuate a planetary gear coupled to an adjustment rod that is coupled to the adjustment knob such that a ring gear of the planetary gear contacts a brake pad coupled to a housing of the circular stapler and friction between the ring gear and the brake pad causes linear actuation of the planetary gear, and detecting the linear actuation of the planetary gear with at least one sensor positioned adjacent to the planetary gear.
